(19) United States
(12) Patent Application Publication
(10) Pub. No.: US 2023/0041494 A1
(43) Pub. Date: Feb. 9, 2023

(54) SYSTEM AND METHOD FOR POWERING HANDHELD INSTRUMENTS FROM A SURGICAL GENERATOR

(71) Applicant: Covidien LP, Mansfield, MA (US)

(72) Inventors: Anthony D. Calderoni, Bristol, CT (US); John M. Pantazis, Stratford, CT (US)

(21) Appl. No.: 17/393,856

(22) Filed: Aug. 4, 2021

(51) Int. Cl. A61B 18/12

(52) U.S. Cl. CPC A61B 18/1206; A61B 2018/1266; A61B 2018/1286

(57) ABSTRACT

A surgical system includes an electrosurgical generator configured to generate an electrosurgical voltage and to output the same through a generator port, an instrument adapter configured to generate a DC voltage from the electrosurgical voltage, and a handheld powered instrument. The instrument adapter includes an adapter plug configured to electrically couple to the generator port and an adapter port configured to output the DC voltage. The handheld powered instrument includes an instrument plug configured to electrically couple to the adapter port to receive the DC voltage.

### BACKGROUND

### Technical Field

The present disclosure relates to handheld powered instruments, and in particular, to a system including an electrosurgical generator configured to power handheld powered instruments.

### Background of Related Art

Handheld powered instruments, such as surgical stapling instruments for stapling tissue during a surgical procedure are well known. Such instruments allow a clinician to fasten tissue more quickly than traditional suturing techniques to shorten the length of the surgical procedure and minimize patient trauma. During a stapling procedure, the instruments typically are powered using bulky power supplies embedded in instruments themselves.

A continuing need exists in the art for a system that powers handheld powered instruments using a surgical generator.

### SUMMARY

In accordance with aspects of the disclosure, a surgical system includes an electrosurgical generator configured to generate an electrosurgical voltage and to output the same through a generator port, an instrument adapter configured to generate a DC voltage from the electrosurgical voltage, and a handheld powered instrument. The instrument adapter includes an adapter plug configured to electrically couple to the generator port and an adapter port configured to output the DC voltage. The handheld powered instrument includes an instrument plug configured to electrically couple to the adapter port to receive the DC voltage.

In an aspect of the present disclosure, the instrument adapter may further include a power circuit configured to generate the DC voltage from the electrosurgical voltage. The power circuit may include a transformer configured to step down the electrosurgical voltage to a second electrosurgical voltage lower than the electrosurgical voltage and a rectifier electrically coupled to the transformer and configured to rectify the second electrosurgical voltage to form the DC voltage for powering the handheld powered instrument.

In another aspect of the present disclosure, the power circuit may include a capacitor disposed across an output of the rectifier, the capacitor configured to reduce ripple on the DC voltage.

In yet another aspect of the present disclosure, the transformer may include a primary coil and a secondary coil.

In a further aspect of the present disclosure, the primary coil may include a Litz wire.

In yet a further aspect of the present disclosure, the instrument adapter may include an indicator configured to indicate an output of DC power.

In an aspect of the present disclosure, the primary coil may have a greater number of turns than the secondary coil.

In another aspect of the present disclosure, the handheld powered instrument may be a surgical stapler.

In yet another aspect of the present disclosure, the handheld powered instrument may include an integrated cable with the instrument plug located on a first end of the integrated cable.

In accordance with aspects of the disclosure, a surgical system includes an electrosurgical generator configured to generate an electrosurgical voltage and to output the same through a generator port and a handheld powered instrument. The handheld powered instrument includes an instrument plug configured to electrically couple to the generator port to receive the electrosurgical voltage and a power circuit configured to generate a DC voltage from the electrosurgical voltage.

In a further aspect of the present disclosure, the power circuit may include a transformer configured to step down an electrosurgical voltage to a second electrosurgical voltage lower than the electrosurgical voltage and a rectifier electrically coupled to the transformer and configured to rectify the second electrosurgical voltage to form the DC voltage for powering the handheld powered instrument.

In yet a further aspect of the present disclosure, the power circuit may further include a capacitor disposed across an output of the rectifier, the capacitor configured to reduce ripple on the DC voltage.

In an aspect of the present disclosure, the transformer may include a primary coil and a secondary coil.

In another aspect of the present disclosure, the primary coil may include Litz wire.

In yet another aspect of the present disclosure, the handheld powered instrument may include an indicator configured to indicate an output of DC voltage from the power circuit.

In a further aspect of the present disclosure, the primary coil may have a greater number of turns than the secondary coil.

In yet a further aspect of the present disclosure, the transformer may include a stepdown transformer.

In yet a further aspect of the present disclosure, the handheld powered instrument may include an integrated cable with the instrument plug located on a first end of the integrated cable.

### BRIEF DESCRIPTION OF THE DRAWINGS

The present disclosure may be understood by reference to the accompanying drawings, when considered in conjunction with the subsequent, detailed description, in which:

FIG. 1 is a perspective view of a surgical system according to an aspect of the present disclosure;

FIG. 2 is a front view of an electrosurgical generator of FIG. 1 according to an aspect of the present disclosure;

FIG. 3 is a schematic diagram of the electrosurgical generator of FIG. 1 according to an aspect of the present disclosure;

FIG. 4 is a block diagram of an instrument adapter of the surgical system of FIG. 1 according to an aspect of the present disclosure; and

FIG. 5 is a printed circuit diagram of a power circuit of the surgical system of FIG. 1 according to an aspect of the present disclosure.

### DETAILED DESCRIPTION

Aspects of the presently disclosed system are described in detail with reference to the drawings, in which like reference numerals designate identical or corresponding elements in each of the several views. As used herein the term “distal” refers to the portion of the surgical instrument coupled thereto that is closer to the patient, while the term “proximal” refers to the portion that is farther from the patient.

In the following description, well-known functions or constructions are not described in detail to avoid obscuring the present disclosure in unnecessary detail. Those skilled in the art will understand that the present disclosure may be adapted for use with either an endoscopic instrument, a laparoscopic instrument, or an open instrument. It should also be appreciated that different electrical and mechanical connections and other considerations may apply to each particular type of instrument.

An electrosurgical generator according to the present disclosure may be used in monopolar and/or bipolar electrosurgical procedures, including, for example, cutting, coagulation, ablation, and vessel sealing procedures. The generator may include a plurality of outputs for interfacing with various ultrasonic and electrosurgical instruments (e.g., ultrasonic dissectors and hemostats, monopolar instruments, return electrode pads, bipolar electrosurgical forceps, footswitches, etc.). Further, the generator may include electronic circuitry configured to generate radio frequency energy specifically suited for powering ultrasonic instruments and electrosurgical instruments operating in various electrosurgical modes (e.g., cut, blend, coagulate, division with hemostasis, fulgurate, spray, etc.) and procedures (e.g., monopolar, bipolar, vessel sealing).

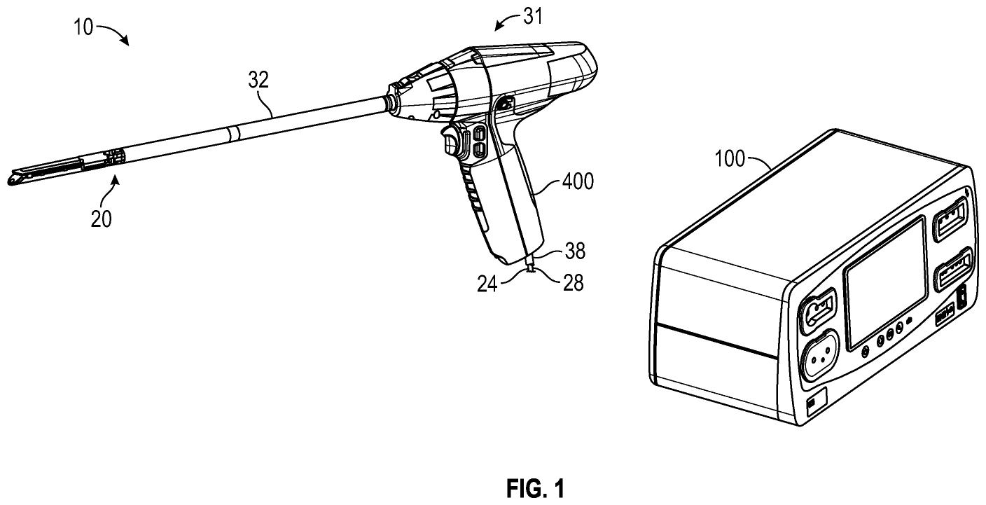

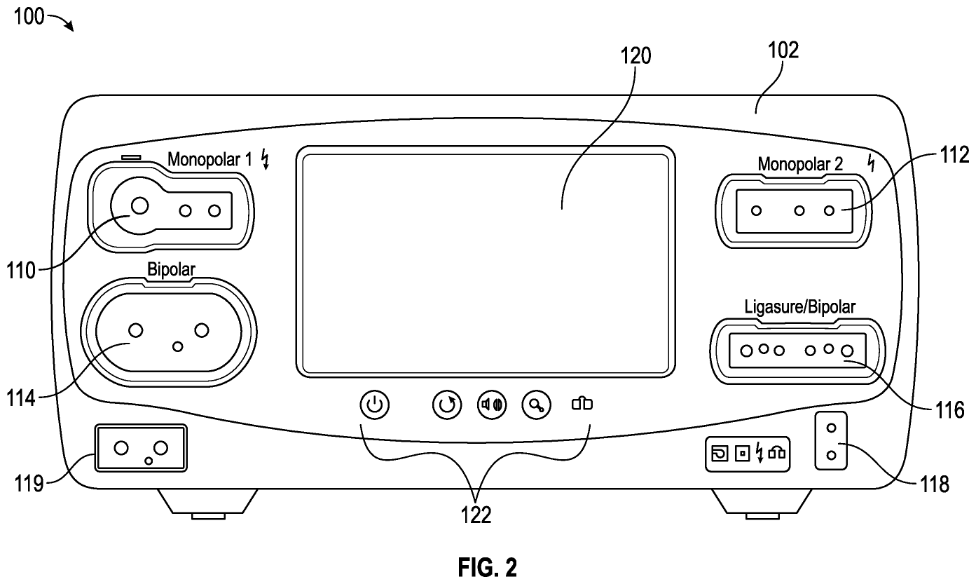

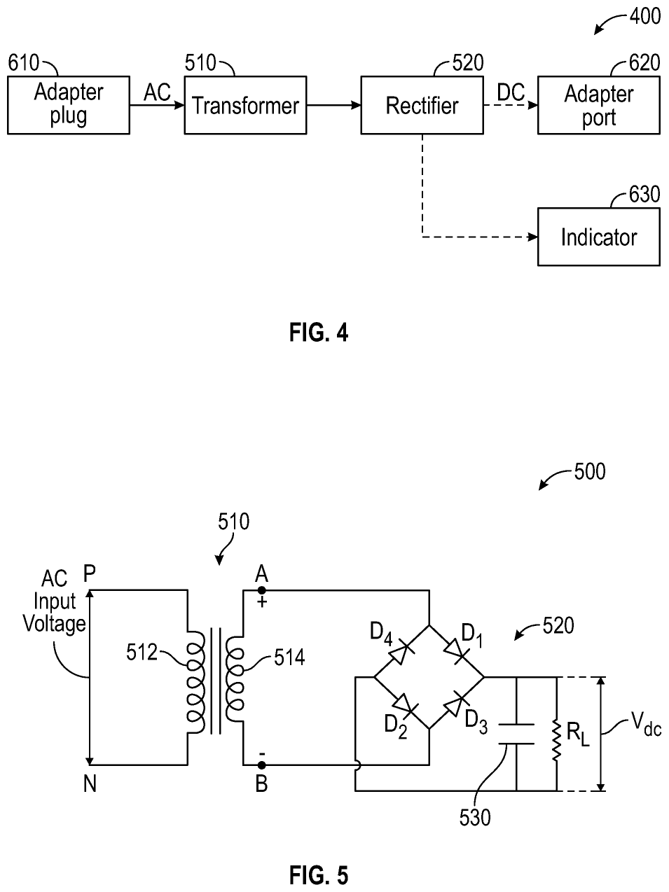

Referring to FIG. 1 a surgical system 10 is shown which includes an electrosurgical generator 100 (FIG. 2) configured to generate an electrosurgical voltage, one or more handheld powered instruments 20 (for example, shown as a surgical stapling instrument) for treating tissue of a patient, and an instrument adapter 400 (FIG. 4) configured to generate a DC voltage for powering the handheld powered instruments 20 from the electrosurgical voltage. This provides the benefit of using a low-cost power source that is readily available in operating rooms.

Electrosurgical alternating radio frequency (hereinafter “RF”) current is supplied to the instrument adapter 400 by the generator 100 through cable 38 that includes supply line 24. The alternating RF current is returned to the generator 100 through cable 38 that includes the return line 28. For a more detailed description of exemplary operation of the stapling instrument see U.S. Pat. No. 9,055,943 (the '943 patent), which discloses a surgical stapling instrument including a power handle assembly having a plurality of motors for actuating a stapler end effector. The entire disclosure of the '943 patent is hereby incorporated by reference herein. Although a linear stapling instrument is shown, it is contemplated that other handheld powered instruments may be used, such as a circular stapling instrument.

The instrument adapter 400 is coupled to the generator 100 at a port having connections to the active and return terminals via an adapter plug 610 disposed at the end of the cable 38, wherein the adapter plug 610 includes contacts from the supply and return lines 24, 28 as described in more detail below.

It is contemplated that the instrument adapter 400, for example, may be housed in a housing 31 of the handheld powered instrument 20, incorporated in the cable 38, and/or may be a standalone device. In aspects, the cable 38 may be integrated into a clamshell (not shown) of the handheld powered instrument 20 as a sterile disposable barrier. The cable 38 may be either disposable or reusable cable.

With reference to FIG. 2, a front face 102 of the generator 100 is shown. The generator 100 may include a plurality of ports 110, 112, 114, 116 to accommodate various types of electrosurgical instruments and a port 118 for coupling to a return electrode pad and a port 119 configured to couple to the footswitch (not shown). The ports 110, 112, 114 and 116 are configured to couple to the handheld electrosurgical and powered instruments (e.g., surgical stapling instrument). The generator 100 includes a display 120 for providing the user with variety of output information (e.g., intensity settings, treatment complete indicators, etc.). The display 120 is a touchscreen configured to display a menu corresponding to each of the ports 110, 112, 114, 116 and the instrument coupled. The user also adjusts inputs by touching corresponding menu options. The generator 100 also includes suitable input controls 122 (e.g., buttons, activators, switches, touch screen, etc.) for controlling the generator 100.

The generator 100 is configured to operate in a variety of modes and is configured to output monopolar and/or bipolar waveforms corresponding to the selected mode. Each of the modes may be activated by a button (not shown) disposed on the handheld powered instrument 20. Each of the modes operates based on a preprogrammed power curve that limits how much power is output by the generator 100 at varying impedance ranges of the load (e.g., tissue). Each of the power curves includes power, voltage and current control ranges that are defined by the user-selected intensity setting and the measured minimum impedance of the load.

The generator 100 may operate in the following monopolar modes, which include, but are not limited to, cut, blend, division with hemostasis, fulgurate and spray. The generator 100 may operate in the following bipolar modes, including bipolar cutting, bipolar coagulation, automatic bipolar which operates in response to sensing tissue contact, and various algorithm-controlled vessel sealing modes. The generator 100 may be configured to deliver energy required to power an ultrasonic transducer. Thereby enabling control and modulation of ultrasonic surgical instruments.

Each of the RF waveforms may be either monopolar or bipolar RF waveforms, each of which may be continuous or discontinuous and may have a carrier frequency from about 200 kHz to about 500 kHz. As used herein, continuous waveforms are waveforms that have a 100% duty cycle. In aspects, continuous waveforms are used to impart a cutting effect on tissue. Conversely, discontinuous waveforms are waveforms that have a non-continuous duty cycle, e.g., below 100%. In aspects, discontinuous waveforms are used to provide coagulation effects to tissue.

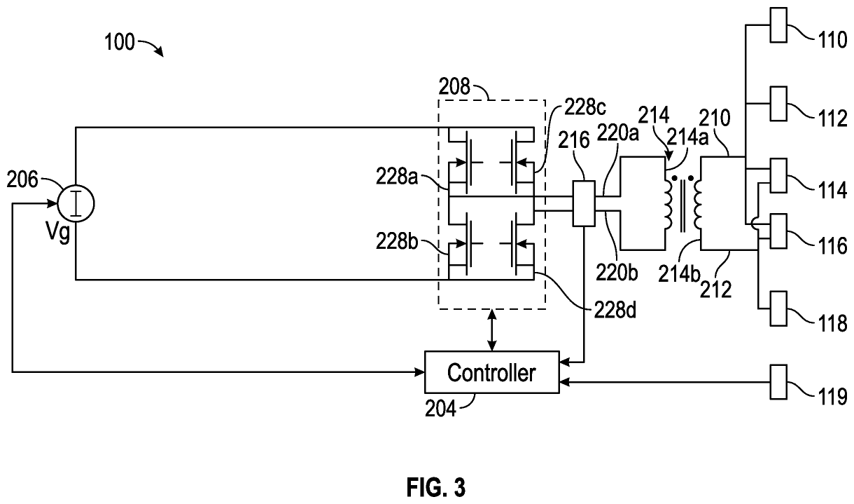

With reference to FIG. 3, the generator 100 includes a controller 204, a power supply 206, and a RF inverter 208. The power supply 206 may be high voltage, DC power supplies connected to a common AC source (e.g., line voltage) and provide high voltage, DC power to their respective RF inverter 208, which then convert DC power into a RF waveform through active terminal 210 and return terminal 212 corresponding to the selected mode. The active terminal 210 and the return terminal 212 are coupled to the RF inverter 208 through an isolation transformer 214. The isolation transformer 214 includes a primary winding 214a coupled to the RF inverter 208 and a secondary winding 214b coupled to the active and return terminals 210 and 212.

Electrosurgical energy for energizing the handheld powered instrument 20 (e.g., a surgical stapling instrument) may be delivered through the ports 110, 112, 114, 116, and 119, each of which is coupled to the active terminal 210. RF energy is returned through the return electrode pad coupled to the port 118, which in turn, is coupled to the return terminal 212. The secondary winding 214b of the isolation transformer 214 is coupled to the active and return terminals 210 and 212. RF energy for energizing a bipolar electrosurgical instrument is delivered through the ports 114 and 116, each of which is coupled to the active terminal 210 and the return terminal 212. The generator 100 may include a plurality of steering relays or other switching devices configured to couple the active terminal 210 and the return terminals 212 to various ports 110, 112, 114, 116, 118 based on the handheld powered instruments being used.

The RF inverter 208 is configured to operate in a plurality of modes, during which the generator 100 outputs corresponding waveforms having specific duty cycles, peak voltages, crest factors, etc. It is envisioned that in other aspects, the generator 100 may be based on other types of suitable power supply topologies. RF inverter 208 may be a resonant RF amplifier or non-resonant RF amplifier, as shown. A non-resonant RF amplifier, as used herein, denotes an amplifier lacking any tuning components, i.e., conductors, capacitors, etc., disposed between the RF inverter and the load, e.g., tissue.

The controller 204 may include a processor (not shown) operably connected to a memory (not shown). The controller 204 is operably connected to the power supply 206 and/or RF inverter 208 allowing the processor to control the output of the RF inverter 208 of the generator 100 according to either open and/or closed control loop schemes. A closed loop control scheme is a feedback control loop, in which a plurality of sensors measures a variety of tissue and energy properties (e.g., tissue impedance, tissue temperature, output power, current and/or voltage, etc.), and provides feedback to the controller 204. The controller 204 then controls the power supply 206 and/or RF inverter 208, which adjust the DC and/or RF waveform, respectively.

The generator 100 according to the present disclosure may also include a plurality of sensors 216, each of which monitors output of the RF inverter 208 of the generator 100. The sensor 216 may be any suitable voltage, current, power, and impedance sensors. The sensors 216 are coupled to leads 220a and 220b of the RF inverter 208. The leads 220a and 220b couple the RF inverter 208 to the primary winding 214a of the transformer 214. Thus, the sensors 216 are configured to sense voltage, current, and other electrical properties of energy supplied to the active terminal 210 and the return terminal 212.

In further aspects, the sensor 216 may be coupled to the power supply 206 and may be configured to sense properties of DC current supplied to the RF inverter 208. The controller 204 also receives input (e.g., activation) signals from the display 120, the input controls 122 of the generator 100 and/or the handheld powered instrument 20. The controller 204 adjusts power outputted by the generator 100 and/or performs other control functions thereon in response to the input signals.

The RF inverter 208 includes a plurality of switching elements 228a-228d, which are arranged in an H-bridge topology. In aspects, RF inverter 208 may be configured according to any suitable topology including, but not limited to, half-bridge, full-bridge, push-pull, and the like. Suitable switching elements include voltage-controlled devices such as transistors, field-effect transistors (FETs), combinations thereof, and the like. In aspects, the FETs may be formed from gallium nitride, aluminum nitride, boron nitride, silicon carbide, or any other suitable wide bandgap materials.

The controller 204 is in communication with the RF inverter 208, and in particular, with the switching elements 228a-228d. Controller 204 is configured to output control signals, which may be pulse-width modulated (“PWM”) signals, to switching elements 228a-228d. In particular, controller 204 is configured to modulate a control signal supplied to switching elements 228a-228d of the RF inverter 208. The control signal provides PWM signals that operate the RF inverter 208 at a selected carrier frequency. Additionally, controller 204 is configured to calculate power characteristics of the output of the RF inverter 208 of the generator 100, and control the output of the generator 100 based at least in part on the measured power characteristics including, but not limited to, voltage, current, and power at the output of RF inverter 208.

With reference to FIG. 4, the instrument adapter 400 is configured to generate a DC voltage from the electrosurgical voltage, which is provided by the generator 100 of the system 10 of FIG. 1. The instrument adapter 400 generally includes an adapter plug 610 disposed at the end of the cable 38. The adapter plug 610 includes contacts from the supply and return lines 24, 28, and a power circuit 500 (FIG. 5) configured to generate the DC voltage from the electrosurgical voltage. The adapter plug 610 of the instrument adapter 400 is configured to electrically couple to one of the generator ports 110, 112, 114, 116, 118, 119. The generator ports 110, 112, 114, 116, 118, 119 include electrical contacts (not shown) configured to couple to the electrical contacts of the adapter plug 610 once the plug 610 is inserted into the port (110, 112, 114, 116, 118, 119) to establish an electrical connection.

In aspects, the instrument adapter 400 may include an adapter port 620 configured for output the DC voltage to the handheld powered instrument 20. The instrument adapter 400 may include an indicator 630, such as an LED and/or a display configured to indicate an output of DC power.

Referring to FIG. 5, the power circuit 500 generally includes a transformer 510 and a rectifier 520. The transformer 510 is configured to step down the electrosurgical voltage to a second electrosurgical voltage lower than the electrosurgical voltage. The transformer 510 includes a primary coil 512 and one or more secondary coil(s) 514. A varying current in the primary coil 512 of the transformer 510 produces a varying magnetic flux in the transformer's core, which induces a varying electromotive force across the secondary coil 514 wound around the same core. The transformer 510 may be a stepdown transformer, where the primary coil has a higher number of turns than the secondary coil. The transformer generally includes a core, a bobbin, and/or a yoke. The core may include a ferrite material and may include an “E core” shape. It is contemplated that other shapes may be used. The core may be comprised of two halves. The bobbin (e.g., a coil former) is composed of plastic (and/or for example, phenolic materials) and metal pins to mount the transformer for example, to a printed circuit board. The yoke may include metal springs configured to hold the two halves of the core together.

The primary and/or secondary coil may be made of Litz wire. As used herein, the term “Litz wire” refers to a type of multi strand wire or cable used for carrying alternating current (AC) at radio frequencies. The wire is designed to reduce the skin effect and proximity effect losses in conductors used at RF frequencies. Litz wire may include a plurality of thin wire strands, individually insulated and twisted or woven together, following one of several predetermined patterns often involving several levels (groups of twisted wires are twisted together, etc.). These winding patterns equalize the proportion of the overall length over which each strand is at the outside of the conductor. This has the effect of distributing the current equally among the wire strands, reducing the resistance.

The rectifier 520 is electrically coupled to the transformer 510 and configured to rectify the second electrosurgical voltage to form the DC voltage for powering the handheld powered instrument 20. The rectifier 520 may include, but is not limited to a bridge rectifier, a half wave rectifier, a full wave rectifier, a single-phase rectifier, push-pull, a three phase rectifier, and/or a voltage multiplying rectifier. In aspects, the power circuit may include a capacitor 530 electrically coupled to an output of the rectifier 520. The capacitor 530 is configured to reduce ripple on the DC voltage. The DC voltage is for powering the motors (not shown), drive electronics (not shown), and/or other functions of the handheld powered instrument 20. The rectifier may include Schottky diodes for rectifying the RF voltages. It is contemplated that additional filtering may be used to suppress RF noise and emissions. For example, the additional filtering may include a passive L-C network.

While several aspects of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope of the claims appended hereto.

What is claimed is:

1. A surgical system comprising:
an electrosurgical generator configured to generate an electrosurgical voltage and to output the same through a generator port;
an instrument adapter configured to generate a DC voltage from the electrosurgical voltage, the instrument adapter including:
an adapter plug configured to electrically couple to the generator port; and
an adapter port configured to output the DC voltage; and
a handheld powered instrument including an instrument plug configured to electrically couple to the adapter port to receive the DC voltage.

2. The surgical system according to claim 1, wherein the instrument adapter further includes:
a power circuit configured to generate the DC voltage from the electrosurgical voltage, the power circuit including:
a transformer configured to step down the electrosurgical voltage to a second electrosurgical voltage lower than the electrosurgical voltage; and
a rectifier electrically coupled to the transformer and configured to rectify the second electrosurgical voltage to form the DC voltage for powering the handheld powered instrument.

3. The surgical system according to claim 2, wherein the power circuit includes a capacitor disposed across an output of the rectifier, the capacitor configured to reduce ripple on the DC voltage.

4. The surgical system according to claim 2, wherein the transformer includes a primary coil and a secondary coil.

5. The surgical system according to claim 4, wherein the primary coil includes a Litz wire.

6. The surgical system according to claim 4, wherein the secondary coil includes a Litz wire.

7. The surgical system according to claim 1, wherein the instrument adapter includes an indicator configured to indicate an output of DC power.

8. The surgical system according to claim 4, wherein the primary coil has a greater number of turns than the secondary coil.

9. The surgical system according to claim 1, wherein the handheld powered instrument is a surgical stapler.

10. The surgical system according to claim 2, wherein the handheld powered instrument includes an integrated cable with the instrument plug located on a first end of the integrated cable.

11. A surgical system comprising:
an electrosurgical generator configured to generate an electrosurgical voltage and to output the same through a generator port; and
a handheld powered instrument comprising:
an instrument plug configured to electrically couple to the generator port to receive the electrosurgical voltage; and
a power circuit configured to generate a DC voltage from the electrosurgical voltage.

12. The surgical system according to claim 11, wherein the power circuit includes:
a transformer configured to step down an electrosurgical voltage to a second electrosurgical voltage lower than the electrosurgical voltage; and
a rectifier electrically coupled to the transformer and configured to rectify the second electrosurgical voltage to form the DC voltage for powering the handheld powered instrument.

13. The surgical system according to claim 12, wherein the power circuit further includes a capacitor disposed across an output of the rectifier, the capacitor configured to reduce ripple on the DC voltage.

14. The surgical system according to claim 12, wherein the transformer includes a primary coil and a secondary coil.

15. The surgical system according to claim 14, wherein the primary coil includes Litz wire.

16. The surgical system according to claim 14, wherein the secondary coil includes Litz wire.

17. The surgical system according to claim 11, wherein the handheld powered instrument includes an indicator configured to indicate an output of DC voltage from the power circuit.

18. The surgical system according to claim 14, wherein the primary coil has a greater number of turns than the secondary coil.

19. The surgical system according to claim 12, wherein the transformer includes a stepdown transformer.

20. The surgical system according to claim 12, wherein the handheld powered instrument includes an integrated cable with the instrument plug located on a first end of the integrated cable.
